(19) United States
(12) Patent Application Publication
(10) Pub. No.: US 2012/0087881 A1
(43) Pub. Date: Apr. 12, 2012

(54) Safe & easy

(76) Inventor: Farouk Al-Salihi, New York, NY (US)

(21) Appl. No.: 12/928,485

(22) Filed: Feb. 17, 2011

Related U.S. Application Data
(60) Provisional application No. 61/404,724, filed on Oct. 8, 2010.

(51) Int. Cl. A61K 8/67; A61Q 15/00

(52) U.S. Cl. 424/67

(57) ABSTRACT

Some members of the public who uses the commercially available under-arm deodorants develop allergy and sensitivity to the under-arm deodorant.

Safe & Easy is made of a simple and very safe chemicals with remote possibility of causing irritation and allergy

our Safe & Easy is to make available to the public who could not use the commercial available under-arm deodorants because of their side effects.

### TECHNICAL FIELD AND INDUSTRIAL APPLICABILITY OF THE INVENTION

This invention related to the use of the under-arm deodorant for public use.

There are many kinds of under-arm deodorant which is commercially available which are made by many companies, the ingredient used include Aluminum zirconium, Tetrachloro hydrex, Cyclopenta Silcoxane, Proplene Carbanate and many others.

The public who use the currently available under arm deodorants some time they devolve sensitivity and allergic, reactions to the ingredient which are used in the preparation of the under-arm deodorant.

The main ingredient used in our invention is Menthol 2%, Menthol is an organic compound naturally found in Mint plants it is also synthetically manufactured it is a waxy crystalline substance clear or white in color the main form of Menthol occurring in nature is(-) Menthol which is assigned the (1R, 2S, 5R) configuration.

Menthol is widely used in number of products, it was first isolated from pepper mint oil in 1771 in the west. Menthol is included in many products for a variety of reasons to relief minor sore throat and minor mouth and throat irritation it is used as antipruritic it is commonly used in oral hygiene products like mouth wash and tooth paste and as a food flavor agent like in chewing gum and candy.

In some people who developed under-arm itching and irritation which developed because of the use of the commercially available under-arm deodorant we found that the use of safe and easy which contain Menthol 2% with zinc oxide and with vitamin A&D cream have removed the effect of the under-arm deodorant and relief the itching and irritation caused by the use of many of the commercially available under-arm deodorant.

The only known under-arm deodorant to be used in the individual who developed irritation and itching due to the use of the commercial available deodorant is our invention “Safe and Easy” which is mixture of Menthol, Zinc oxide and with vitamin A&D cream.

All medicines may causes side effects but many people have no, or minor side effects. NO COMMON side effects have been reported with Menthol/Zinc oxide powder and with vitamin A&D cream.

I claim:

1. The safe and easy is under arm deodorant to be used by the people who are allergic where by using other commercially available under arm deodorant.

2. The safe and easy of claim 1 wherein said that in safe and easy the mixture used contain are menthol 0.2% zinc oxide powder and vitamin a and d cream.

3. The safe and easy of claim 2 wherein review of the literature speaking with several pharmacists and using computer data non of the items used in safe and easy mixtures ever been claimed before that were used as under arm deodorant.

4. The safe and easy of claim 1 wherein to be used in the Area of arm pit maxilla as needed or after taking Shower as an under arm deodorant.
